(19) United States
(12) Patent Application Publication
(10) Pub. No.: US 2011/0044930 A1
(43) Pub. Date: Feb. 24, 2011

(54) COPOLYHYDROXYALKYLGLUTAMINES FUNCTIONALISED WITH HYDROPHOBIC GROUPS, AND USES THEREOF, ESPECIALLY IN THERAPEUTICS

(76) Inventors: Rémi Soula, Lyon (FR); You-Ping Chan, Ternay (FR)

Correspondence Address: PATTON BOGGS LLP, 8484 WESTPARK DRIVE, SUITE 900, MCLEAN, VA, 22102, US

(21) Appl. No.: 11/883,223

(22) PCT Filed: Jan. 23, 2006
(86) PCT No.: PCT/EP2006/050369

§ 371 Date: Oct. 29, 2010

(30) Foreign Application Priority Data
Jan. 27, 2005 (FR) 05 50231

(51) Int. Cl. A61K 31/765; C08G 69/10

(52) U.S. Cl. 424/78.17; 525/54.1

(57) ABSTRACT

The invention relates to novel biodegradable materials which are based on modified polyamino acids and which can be used for the vectorisation of active principle(s) (AP). The invention also relates to novel pharmaceutical, cosmetic, dietary or phytosanitary compositions based on said polyamino acids. The aim of the invention is to provide a novel polymer raw material which can be used for the vectorisation of active principles and which can optimally fulfil all required specifications in said area, namely: biocompatibility, biodegradability and the ability to become easily associated with many active principles or to solubilise said principles and to release same in vivo. Said aim is achieved with novel copolyhydroxyalkylglutamines comprising glutamine units and optionally glutamate units and bearing hydrophobic groups containing between 8 and 30 carbon atoms. Said copolyhydroxyalkylglutamines are amphiphilic and can be easily and economically transformed into particles for the vectorisation of active principles, whereby said particles can form stable aqueous colloidal suspensions.

The present invention relates to novel biodegradable materials based on copolyamino acids, which are useful especially for the vectorization of active principles (AP).

The invention is also directed toward novel pharmaceutical, cosmetic, dietetic or plant-protection compositions based on these modified polyamino acids. These compositions may be of the type that allow the vectorization of APs and that are preferably in the form of emulsions, micelles, particles, gels, implants or films.

The APs under consideration are advantageously biologically active compounds that may be administered to a human or animal body via the oral, parenteral, nasal, vaginal, ocular, subcutaneous, intravenous, intramuscular, intradermal, intraperitoneal, intracerebral, buccal, etc. route.

The APs to which the invention more particularly, but not exclusively, relates are proteins, glycoproteins, peptides, polysaccharides, lipopolysaccharides, oligonucleotides, polynucleotides and organic molecules. However, they may also be cosmetic products or plant-protection products, such as herbicides, insecticides, fungicides, etc.

In the field of vectorization of active principles, especially medicinal active principles, there is a need in many cases:
to protect them against degradation (hydrolysis, on-site precipitation, enzymatic digestion, etc.) until they reach their site of action,
and/or to control their rate of release so as to maintain a therapeutic level over a defined period,
and/or to convey them (while protecting them) to the site of action.

For these purposes, several types of polymers have been studied and certain polymers are even commercially available. Examples that may be mentioned include polymers of the polylactic, polylactic-glycolic, polyoxyethylene-oxypropylene, polyamino acid or polysaccharide type. These polymers constitute starting materials for manufacturing, for example, bulk implants, microparticles, nanoparticles, vesicles, micelles or gels. Besides the fact that these polymers should be suited to the manufacture of such systems, they should also be biocompatible, nontoxic, nonimmunogenic and economical and they should be able to be removed easily from the body and/or be biodegradable. Regarding this last aspect, it is furthermore essential for the biodegradation in the body to generate nontoxic products.

Another aspect that is also important in the development of an associative polymer is its water solubility. The possibility of dissolving a large amount of polymer makes it possible to have a polymer/active principle ratio that is suited to the desired release profile.

As illustrations of the prior art concerning polymers used as starting materials for preparing AP vectorization systems, various patents or patent applications or scientific articles are mentioned hereinbelow.

Patent U.S. Pat. No. 4,652,441 describes polylactide microcapsules encapsulating the hormone LH-RH. These microcapsules are produced by preparing a water-in-oil-in-water emulsion comprising an aqueous inner layer containing the hormone, a substance (gelatin) that fixes said hormone, an oily layer of polylactide, and also an aqueous outer layer (polyvinyl alcohol). The release of the AP may take place over a period of more than two weeks after subcutaneous injection.

Patent U.S. Pat. No. 6,153,193 describes compositions based on amphiphilic poly(oxyethylene)-poly(oxypropylene) micelles for the vectorization of anticancer agents such as adriamycin.

Akiyoshi et al. (J. Controlled Release 1998, 54, 313-320) describe pullulans that are made hydrophobic by grafting cholesterol thereon, and that form nanoparticles in water. These nanoparticles capable of reversibly complexing with insulin form stable colloidal suspensions.

Patent U.S. Pat. No. 4,351,337 describes amphiphilic copolyamino acids based on leucine and glutamate, which may be used in the form of implants or microparticles for the controlled release of active principles. The release of these active principles may take place over a very long period depending on the rate of degradation of the polymer.

Patent U.S. Pat. No. 4,888,398 describes polymers based on polyglutamate or polyaspartate, and optionally polyleucine, with pendent groups of alkyloxycarbonylmethyl type, placed randomly on the polyamino acid chain. These polyamino acids, grafted with side groups e.g. methoxycarbonylmethyl, may be used in the form of biodegradable implants containing a sustained-release AP.

Patent U.S. Pat. No. 5,904,936 describes nanoparticles obtained from a polyleucine-polyglutamate block polymer, which are capable of forming stable colloidal suspensions and of spontaneously combining with biologically active proteins without denaturing them. Said proteins may then be released in vivo in a controlled manner, over a long period.

Patent U.S. Pat. No. 5,449,513 describes amphiphilic block copolymers comprising a polyoxyethylene block and a polyamino acid block, for example poly(beta-benzyl L-aspartate). These polyoxyethylene-polybenzyl aspartate polymers form micelles that are capable of encapsulating hydrophobic active molecules such as adryamycin or indomethacin.

Patent application WO-A-99/61512 describes polylysines and polyornithines functionalized with a hydrophobic group (palmitic acid linked to the polylysine or ornithine) and a hydrophilic group (polyoxyethylene). These polymers, for example polylysine grafted with polyoxyethylene and palmitoyl chains, form, in the presence of cholesterol, vesicles capable of encapsulating doxorubicin or DNA. These polylysine-based polymers are cationic in physiological medium.

Patent U.S. Pat. No. 6,630,171 from the Applicant describes block or random poly(sodium glutamate)-poly(methyl, ethyl, hexadecyl or dodecyl glutamate) polymers that are capable of forming stable colloidal suspensions and of spontaneously combining with biologically active proteins without denaturing them. These proteins may then be released in vivo in a controlled manner, over a long period. These amphiphilic linear copolyamino acids are modified by the presence of a hydrophobic alkyl side chain. These alkyl groups are covalently grafted onto the polymer via an ester function. These polymers are anionic in physiological medium.

In the same field, the Applicant has described in several patent applications polyglutamate-based polymers (anionic) with related concepts.

Patent application WO-A-03/104 303 describes anionic polyamino acids functionalized with alpha tocopherol.

Patent application WO-A-2004/013 206 describes anionic polyamino acids comprising hydrophobic groups and characterized in that these groups are linked to the polymer via a connecting group containing two amide functions, and more specifically via a spacer of lysine or ornithine type.

Patent application WO-A-2004/060 968 describes poly-amino acids functionalized with at least one oligoamino acid group based on leucine and/or isoleucine and/or valine and/or phenylalanine.

Patent application WO-A-87/03891 describes linear, branched or star amphiphilic polymers, with at least two hydrophobic groups linked only at their ends. Said patent application essentially concerns neutral hydrophilic polymers based on polyethylene glycol, as evidenced by all the examples of said patent. However, this type of polymer is not biodegradable, which constitutes a major drawback.

Patent applications WO-A-02/098 951 and WO-A-02/098 952 describe polyalkylglutamines with one or two hydrophobic groups at one end of the polymer. These polymers are capable of forming liposomes and of encapsulating small water-soluble molecules (active principle).

Patent application WO-A-03/002 096 describes polyhydroxyethylaspartamides containing both a polyethylene glycol chain at one end of the polymer and pendent hydrophobic groups. These polymers are capable of forming nanoparticles and of encapsulating active principles.

Patent application WO-A-02/28521 describes a suspension of biocompatible particles for vectorizing (VP) active principles (AP). These VPs are based on a neutral hydrophilic polyamino acid (polyNIAA)/neutral hydrophobic polyamino acid (polyNOAA) diblock copolymer, for example POLY[(LEU)-BLOCK-(GLN-N-HYDROXYETHYL)]ₓ. These polyNIAA/polyNOAA particles are capable of combining in colloidal suspension, in undissolved form, at least one AP and of releasing it, especially in vivo, in a sustained and/or delayed manner. The invention is also directed toward a pulverulent solid from which the VPs are derived and also to the preparation of this solid and of this suspension of polyNIAA/polyNOAA-based VP. These novel VPs form spontaneously, and without the aid of surfactants or organic solvents, stable aqueous suspensions. The invention also relates to VPs in dry form, to the process for preparing them and also to pharmaceutical compositions (in dry or suspension form) comprising these VPs combined with an active principle.

Thus, although very many technical solutions are developed and proposed in the prior art for the vectorization of medicinal active principles, the answer to all the requirements is difficult to obtain and remains to be improved. More specifically, the invention relates to known biodegradable polyamino acids, which may be converted into colloidal vectorization nanoparticles or microparticles capable of reversibly combining with active principles.

In this context, one of the essential objectives of the present invention is to provide novel linear or branched and essentially neutral amphiphilic copolyamino acids, which are soluble over a wide pH range.

These polymers represent an improvement compared with those described in the patents or patent applications mentioned above, in terms of vectorization of an active principle such as a therapeutic protein.

Another essential objective of the present invention is that these polymers should be able to be used for the vectorization of APs and should allow all the specification points to be optimally satisfied, i.e. especially:
capacity:
   to easily and economically form stable aqueous colloidal suspensions,
   to combine easily with many active principles, and
   to release these active principles in vivo,
biocompatibility,
biodegradability,
stability to hydrolysis.

This objective, among others, is achieved by the present invention, which firstly concerns a copolyhydroxyalkylglutamine comprising a plurality of pendent and identical or different hydrophobic groups (HG).

For the purposes of the invention, the term “plurality” means that the copolyhydroxyalkylglutamine comprises, on average, at least two pendent HGs per molecule. It is possible in accordance with the invention for the copolyhydroxyalkylglutamine to contain, in addition to the pendent HGs, HGs attached to at least one of the ends of the copolymer chains.

According to one preferred mode of the invention, this copolyhydroxyalkylglutamine comprises on average at least 3 hydrophobic groups (HG) per copolymer chain.

The copolyhydroxyalkylglutamine also bears hydroxyalkylamine groups. These hydroxyalkylamine groups are preferably linked to the copolymer via an amide bond.

The Applicant has, to its credit, developed a novel family of “essentially neutral” polyhydroxyalkylglutamine-based copolymers functionalized with a plurality of hydrophobic groups and capable of forming stable colloidal systems. The capacity to modify the number of anionic charges on the surface of a colloid makes it possible especially to modify their inter-action with proteins and/or live cells, thus making it possible to vary their bioavailability (see, for example, the article by Furumoto et al. J. Controlled Release 2004, 97, 133-141).

These novel polymers have proved to be particularly suitable for vectorizing proteins. Furthermore, they are easily degraded, in the presence of enzymes, into nontoxic catabolites/metabolites (amino acids).

For the purposes of the invention and throughout the present specification, the terms “association” and “associate” used in order to qualify the relationships between one or more active principles and the copolyhydroxyalkylglutamines mean in particular that the active principle(s) is (are) linked to the copolyhydroxyalkylglutamine(s) especially via a hydrophobic interaction, and/or are encapsulated by the copolyhydroxyalkylglutamine(s).

The hydroxyalkylamine groups that may be used to functionalize the glutamate units of the copolyhydroxyalkylglutamine are identical or different and are chosen, for example, from the following groups: 2-hydroxyethylamine, 3-hydroxypropylamine, 2,3-di-hydroxypropylamine, tris(hydroxymethyl)aminomethane and 6-hydroxyhexylamine.

Advantageously, at least one of the hydrophobic groups HG is included in a hydrophobic graft comprising at least one spacer connecting group (or unit) (“spacer”) for connecting the hydrophobic group HG to a copolyglutamate chain (for example a backbone-copolyglutamate main chain). This connecting group may comprise, for example, at least one direct covalent bond and/or at least one amide bond and/or at least one ester bond. For example, the connecting group may be of the type belonging to the group especially comprising: “amino acid” units other than the constituent monomer unit of the copolyglutamate, amino alcohol derivatives, polyamine derivatives (for example diamines), polyol derivatives (for example diols) and hydroxy acid derivatives.

The grafting of the HGs to the copolyglutamate or polyalkylglutamine chain may be performed by using HG precursors that are capable of binding to the copolyglutamate or copolyhydroxyalkylglutamine chain.

The HG precursors are in practice, without this being limiting, chosen from the group comprising alcohols and amines, since these compounds may be readily functionalized by a person skilled in the art. The grafting of the HGs is explained in greater detail hereinbelow in the description of the process for producing the modified polyamino acids according to the invention.

According to one preferred characteristic, the hydrophobic group HG of the hydrophobic graft contains from 8 to 30 carbon atoms.

→ These hydrophobic groups HG are advantageously and judiciously selected from the group comprising:
linear or branched C8 to C30 alkyls possibly containing at least one unsaturation and/or at least one heteroatom,
C8 to C30 alkylaryls or arylalkyls possibly containing at least one unsaturation and/or at least one heteroatom, and
C8 to C30 (poly)cyclics possibly containing at least one unsaturation and/or at least one heteroatom.

The connecting groups forming with the HGs hydrophobic grafts may be di-, tri- or tetravalent (or even pentavalent and more). In the case of a divalent connecting group, the hydrophobic graft comprises only one HG group, whereas a trivalent connecting group gives the hydrophobic graft bifid nature, i.e. the graft has two HG “feet”. Examples of trivalent connecting groups that may be mentioned, inter alia, include “amino acid” units, for example “glutamic acid” or polyol residues, for example glycerol. Thus, two advantageous but nonlimiting examples of hydrophobic grafts comprising bifid HGs are dialkylglycerols and dialkyl glutamates.

The hydrophobic groups HG may be derived, for example, from groups chosen from the group comprising:

octanol, dodecanol, tetradecanol, hexadecanol, octadecanol, oleyl alcohol, tocopherol or cholesterol.

Preferably, the backbone of the copolyglutamate according to the present invention comprises alpha-L-glutamate and/or alpha-L-glutamic units.

Even more preferably, the copolyhydroxyalkylglutamines according to the invention correspond to one of the general formulae (I) below:

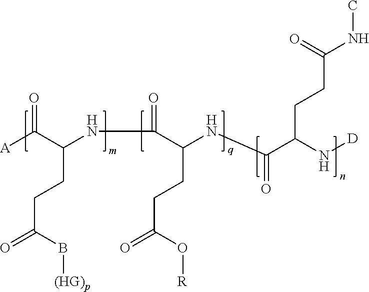

in which
A independently represents:
   a group NHR² in which R² represents an H, a linear C2 to 010 or branched C3 to 010 alkyl or a benzyl,
   a terminal amino acid unit linked via the nitrogen and the acid function(s) of which is (are) optionally modified with an amine or an alcohol corresponding to the definitions NHR² and OR², respectively;
B is a divalent, trivalent or tetravalent bonding group preferably chosen from the following radicals:
—O—, —NH—, —N—(C1 to C5)alkyl-, an amino acid residue (preferably of a natural amino acid), a diol, a triol, a diamine, a triamine, an amino alcohol or a hydroxy acid containing from 1 to 6 carbon atoms;
C is a mono-, di or trihydroxy(C1 to C6)alkyl group, preferably hydroxyethyl, hydroxypropyl or trishydroxymethylmethane;
D represents an H, a linear C2 to C10 or branched C3 to C10 acyl group or a pyroglutamate;
the hydrophobic groups HG represent, independently of each other, a radical chosen from:
   linear or branched C8 to C30 alkyls possibly containing at least one unsaturation and/or at least one heteroatom (preferably O and/or N and/or S), or
   C8 to C30 alkylaryls or arylalkyls possibly containing at least one unsaturation and/or at least one heteroatom (preferably O and/or N and/or S), or
   C8 to C30 (poly)cyclics possibly containing at least one unsaturation and/or at least one heteroatom (preferably O and/or N and/or S);
R represents an H or a cationic species preferably selected from the group comprising:
   metallic cations advantageously chosen from the subgroup comprising: sodium, potassium, calcium, magnesium;
   organic cations advantageously chosen from the subgroup comprising:
      amine-based cations,
      oligoamine-based cations,
      polyamine-based cations (polyethyleneimine being particularly preferred),
      amino acid-based cations advantageously chosen from the class comprising cations based on lysine or arginine,
   or cationic polyamino acids advantageously chosen from the subgroup comprising polylysine or oligolysine;
m, n and q are positive integers;
(m)/(m+q+n) is defined as the molar degree of grafting of the hydrophobic groups HG and ranges from 0.5 up to 90 mol % on condition that each copolymer chain contains on average at least 3 hydrophobic grafts;
(m+q+n) ranges from 10 to 1000 and preferably between 30 and 500;
(q)/(m+q+n) ranges from 0 to 60 mol %;
p is an integer ranging from 1 to 3.

Preferably, the hydrophobic groups HG are randomly distributed.

It is moreover preferable for the molar degree of grafting with hydrophobic units of the copolyhydroxyalkylglutamines according to the invention to be between 2% and 100% and preferably between 5% and 50% on condition that each polymer chain contains on average at least 3 hydrophobic grafts.

The ratio (q)/(m+q+n) of the copolyhydroxyalkylglutamines according to the invention means that they may contain from 0 to about 60 mol % of carboxylic or carboxylate functions.

According to another noteworthy characteristic of the invention, the polymers according to the invention have a molar mass of between 2000 and 200 000 g/mol and preferably between 5000 and 100 000 g/mol.

According to one variant, the copolyhydroxyalkylglutamine according to the invention may bear at least one graft of polyalkylene (preferably ethylene) glycol type linked to a glutamate unit.

Naturally, the invention also covers mixtures of polyamino acids modified as defined above.

Quite remarkably, the copolyhydroxyalkylglutamines of the invention may be used in several ways depending on the nature of the hydrophobic groups and the degree of polymerization of the copolyglutamate. The methods for forming a polymer for the encapsulation of an active principle in the various forms targeted by the invention are known to those skilled in the art. For further details, reference may be made, for example, to these particularly pertinent selected references:
“Microspheres, Microcapsules and Liposomes; vol 1. Preparation and chemical applications” Ed. R. Arshady, Citus Books 1999. ISBN: 0-9532187-1-6.
“Sustained-Release Injectable Products” Ed. J. Senior and M. Radomsky, Interpharm Press 2000. ISBN: 1-57491-101-5.
“Colloidal Drug Delivery Systems” Ed. J. Kreuter, Marcel Dekker, Inc. 1994. ISBN: 0-8247-9214-9.
“Handbook of Pharmaceutical Controlled Release Technology” Ed. D. L. Wise, Marcel Dekker, Inc. 2000. ISBN: 0-8247-0369-3.

These copolyhydroxyalkylglutamines are also extremely advantageous due to the fact that, depending on the length of the copolymer (degree of polymerization) and the nature of the hydrophobic groups, they disperse in water at pH 7.4 (for example with a phosphate buffer) to give colloidal solutions or suspensions or structured or unstructured gels, depending on the copolymer concentration. Furthermore, the copolyhydroxyalkylglutamines (in the form of particles or otherwise) may encapsulate or combine readily with active principles such as proteins, peptides or small molecules. The preferred forming operation is that described in patent U.S. Pat. No. 6,630,171 from the Applicant, which consists in dispersing the copolymer in water and in incubating the solution in the presence of an active principle (AP). This colloidal solution of vectorization particles consisting of copolyhydroxyalkylglutamines according to the invention may then be filtered through a 0.2 μm filter and then directly injected into a patient.

When the hydrophilic/hydrophobic ratio decreases, the copolymer may then form microparticles capable of associating or of encapsulating APs. In this context, the forming of the microparticles may take place by codissolving the AP and the polymer in a suitable organic solvent and then the mixture precipitated in water. The particles are then recovered by filtration and may then be used for an oral administration (in the form of a gel capsule, in compacted and/or coated form or alternatively in a form dispersed in an oil) or for parenteral administration after redispersing in water.

According to one variant, the copolymer may be dissolved in a biocompatible solvent such as N-methylpyrrolidone, ethanol or a suitable oil such as Myglyol® and then injected intramuscularly or subcutaneously or into a tumor. Diffusion of the solvent or of the oil leads to precipitation of the copolymer at the site of injection and thus forms a deposit. These deposits then give controlled release by diffusion and/or erosion and/or hydrolytic or enzymatic degradation of the copolymer.

Independently of the fact that the microparticulate form of the polymer according to the invention is preferred, the copolymers of the invention, in neutral or ionized form, may more generally be used alone or in a liquid, solid or gel composition and in an aqueous or organic medium.

It should be understood that the copolyglutamine-based copolymer contains carboxylic residual functions that are either neutral (COOH form) or ionized (COO⁻ anion), depending on the pH and the composition. In aqueous solution, the counter-cation may be a metal cation such as sodium, calcium or magnesium, or an organic cation such as triethanolamine, tris(hydroxymethyl)aminomethane or a polyamine such as polyethyleneimine.

The copolymers of the invention are obtained, for example, according to methods known to those skilled in the art. Firstly, it is recalled that to obtain a polyamino acid of alpha type, the most common technique is based on the polymerization of N-carboxyamino acid (NCA) anhydrides, which are described, for example, in the article “Biopolymers”, 1976, 15, 1869 and in the book by H. R. Kricheldorf “alpha-Aminoacid-N-carboxy Anhydride and related Heterocycles” Springer Verlag (1987). The NCA derivative is preferably NCA-Glu-O-Bz (Bz=benzyl), since the benzyl group may be selectively hydrolyzed without affecting other chemical functions of the homopolymers or of the hydrophobic group.

A certain number of polymers that may be used according to the invention, for example of poly(alpha-L-glutamic), poly(alpha-D-glutamic), poly(alpha-D,L-glutamate) and poly(gamma-L-glutamic) type of variable masses are commercially available.

Preferably, the copolymers of the invention are synthesized according to two routes. In the first; the hydroxyalkylamine (for example ethanolamine) group and the group B-HG (for example dodecylamine) are first grafted simultaneously or sequentially onto a poly(L-glutamic acid). This reaction may take place in a solvent such as DMF, DMSO or NMP according to the following scheme:

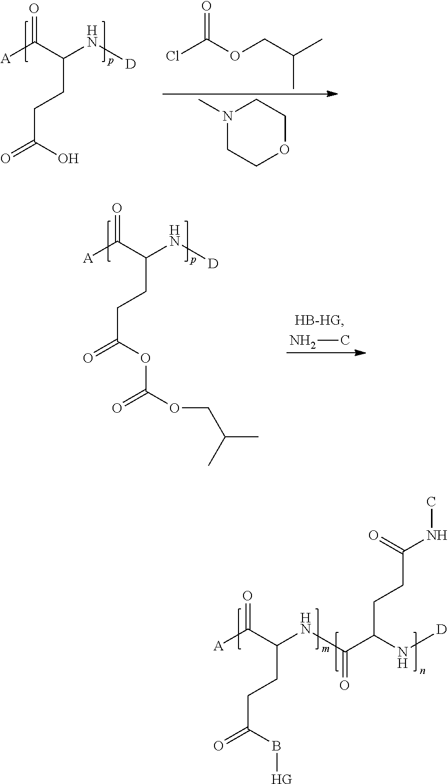

The poly(L-glutamic acid) may be synthesized according to the route described in patent application FR-A-2 801 226. When the group HB-HG is linked via an ester function, it is easier first to graft the group B-HG via a standard coupling reaction using a carbodiimide before grafting the alkylamine.

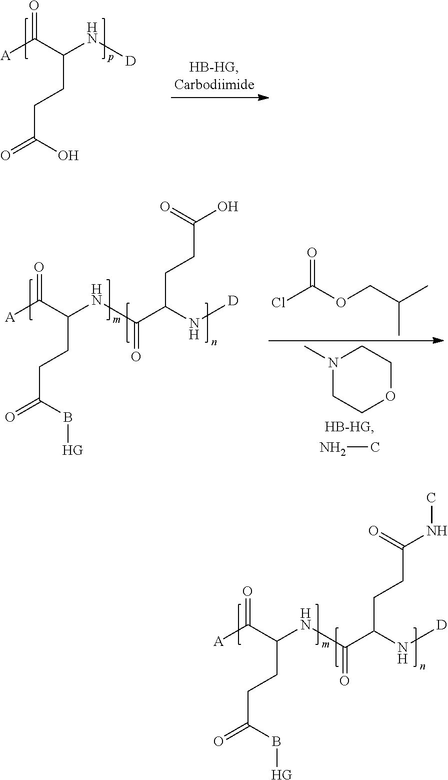

In the second, a poly(alkyl-L-glutamine) is first prepared according to a route described in the literature (see, for example WO-A-02/098 951) and the hydrophobic group HG is grafted onto the OH groups of the alkylamide of the polymer.

The polymerization chemistry and the coupling reactions of the groups are standard and well known to those skilled in the art (see, for example, the patents or patent applications from the Applicant mentioned previously).

These methods will be understood more clearly through the description of the examples.

It should be observed that the degree of polymerization is defined as the molar ratio of the initiator to that of the monomer.

The coupling of the hydrophobic graft HG with an acid function of the polymer is readily performed by reacting the polyamino acid in the presence of a carbodiimide as coupling agent and, optionally, a catalyst such as 4-dimethylaminopyridine and in a suitable solvent such as dimethylformamide (DMF), N-methylpyrrolidone (NMP) or dimethylsulfoxide (DMSO). The carbodiimide is, for example, dicyclohexyl-carbodiimide or diisopropylcarbodiimide. Coupling reagents such as chloroformates may also be used for the formation of amide bonds (see, for example, the book by Bodanszky “Principles of Peptide Synthesis” Springer Verlag 1984 for examples of coupling agents). The degree of grafting is controlled chemically by the stoichiometry of the constituents and reagents or the reaction time. The hydrophobic grafts functionalized with an amino acid other than that of the polymer are obtained by standard peptide coupling or by direct acid-catalyzed condensation. These techniques are well known to those skilled in the art.

According to another of its aspects, the invention is directed toward a pharmaceutical, cosmetic, dietetic or plant-protection composition comprising at least one copolyhydroxyalkylglutamine as defined above and optionally at least one active principle, which may be a therapeutic, cosmetic, dietetic or plant-protection active principle.

According to one advantageous arrangement of the invention, the active principle is combined with the polyamino acid(s) modified with one or more bonds other than one (or more) covalent chemical bond(s). The techniques for combining one or more APs with the modified polyamino acids according to the invention are described especially in patent U.S. Pat. No. 6,630,171. They consist in incorporating at least one active principle into the liquid medium containing Vectorization Particles (VP), so as to obtain a colloidal suspension of VPs containing or combined with one or more active principle(s) AP. This incorporation, which leads to trapping of AP by the VPs, may be performed in the following manner:
dissolving AP in aqueous solution, followed by adding the VPs, either in the form of a colloidal suspension or in the form of isolated VPs (lyophilizate or precipitate);
or adding AP, either as a solution or in pure or preformulated form, to a colloidal suspension of VP particles, optionally prepared beforehand by dispersing the dry VPs in a suitable solvent, such as water.

Preferably, the active principle is a protein, a glycoprotein, a protein linked to one or more polyalkylene glycol chains (preferably polyethylene glycol (PEG): “protein-PEGylated”), a polysaccharide, a liposaccharide, an oligonucleotide, a polynucleotide or a peptide.

According to one variant, the active principle is a “small” hydrophobic, hydrophilic or amphiphilic organic molecule.

For the purposes of the present specification, a “small” molecule is especially a nonprotein small molecule.

As examples of APs that may be combined with the polyamino acids according to the invention, whether or not they are in the form of (nano or micro) particles, mention may be made of:
proteins such as insulin, interferons, growth hormones, interleukins, erythropoietin or cytokines;
peptides such as leuprolide or cyclosporine;
small molecules such as those belonging to the family of anthracyclins, taxoids or camptothecins;
and mixtures thereof.

According to one embodiment, the composition of the invention is in the form of a gel, a solution, a suspension, an emulsion, micelles, nanoparticles, microparticles, an implant, a powder or a film.

According to one of its particularly preferred forms, the composition, containing or not containing active principle(s), is a stable colloidal suspension of polyamino acid micelles and/or microparticles and/or nanoparticles, in an aqueous phase.

According to another embodiment, the composition of the invention is in the form of a solution in a biocompatible solvent and may be injected subcutaneously, intramuscularly or into a tumor.

The composition according to the invention, when it is pharmaceutical, may be administered via the oral, parenteral, nasal, vaginal, ocular, subcutaneous, intravenous, intramuscular, intradermal, intraperitoneal, intracerebral or buccal route.

It may also be envisioned for the composition to be in the form of a solution in a biocompatible solvent or mixture of solvents, which can be injected subcutaneously, intramuscularly or into a tumor.

According to another embodiment, the composition may optionally contain an excipient to adjust the pH and/or the osmolarity and/or to improve the stability (antioxidants) and/or as an antimicrobial agent. These excipients are well known to those skilled in the art (see the book: Injectable Drug Development, P. K. Gupta et al. Interpharm Press, Denver, Colo. 1999).

According to another variant, the composition according to the invention is formulated such that it is capable of forming a deposit at the site of injection. The deposition may be, for example, at least partly brought about by a physiological protein present in vivo.

The invention is also directed toward compositions comprising polyamino acids according to the invention and active principles and that may be used for the preparation of:
medicaments, in particular for oral, nasal, vaginal, ocular, subcutaneous, intravenous, intramuscular, intradermal, intraperitoneal or intracerebral administration, the active principles of these medicaments possibly being, especially, proteins, glycoproteins, proteins linked to one or more polyalkylene glycol chains {for example polyethylene glycol (PEG), in which case they are referred to as “PEGylated” proteins}, peptides, polysaccharides, liposaccharides, oligonucleotides, polynucleotides and small hydrophobic, hydrophilic or amphiphilic organic molecules;
and/or nutrients;
and/or cosmetic or plant-protection products.

According to yet another of its aspects, the invention is directed toward a process for preparing:
medicaments, in particular for oral, nasal, vaginal, ocular, subcutaneous, intravenous, intramuscular, intradermal, intraperitoneal or intracerebral administration, the active principles of these medicaments possibly being, especially, proteins, glycoproteins, proteins linked to one or more polyalkylene glycol chains {for example polyethylene glycol (PEG), in which case they are referred to as “PEGylated” proteins}, peptides, polysaccharides, liposaccharides, oligonucleotides, polynucleotides and small hydrophobic, hydrophilic or amphiphilic organic molecules;
and/or nutrients;
and/or cosmetic or plant-protection products;
   this process being characterized in that it consists essentially in using at least one homopolyamino acid as defined above and/or the composition also as defined above.

The invention also relates to a therapeutic treatment method that consists essentially in administering the composition as described in the present specification, via the oral, parenteral, nasal, vaginal, ocular, subcutaneous, intravenous, intramuscular, intradermal, intraperitoneal, intracerebral or buccal route.

According to one particular variant of the invention, this therapeutic treatment method consists essentially in placing the composition as described above in the form of a solution in a biocompatible solvent and then injecting it subcutaneously, intramuscularly or into a tumor, preferably so as to form a deposit at the site of injection.

The invention will be understood more clearly and its advantages and implementation variants will emerge more clearly from the examples that follow and that describe the synthesis of the polymers of the invention, their conversion into an AP vectorization system (stable aqueous colloidal suspension) and the demonstration of the capacity of such a system to combine with a protein to form pharmaceutical compositions.

### EXAMPLES

### Example 1

### Synthesis of Polymer (1), pHEG C12

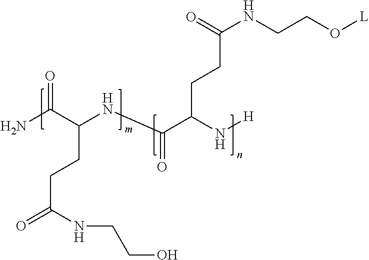

Indices and groups: m=102, n=18, L=COC₁₁H₂₃ (lauroyl) g of a poly(glutamic acid) with a degree of polymerization (DP) of 120 are dissolved, by heating to 80° C., in 77 ml of DMF in a 250 ml three-necked round-bottomed flask. 6.35 g of isobutyl chloroformate and then 5.33 g of N-methylmorpholine are added to this solution cooled to −15° C. The reaction medium is stirred for 15 minutes while allowing the temperature to rise to 0° C. The reaction medium is again cooled to −15° C., followed by addition of a solution of the tosyl salt of 2-aminoethyl laurate in DMF (2.58 g in 25 ml). The reaction medium is stirred for 30 minutes while allowing the temperature to rise to 0° C. The reaction medium is again cooled to −15° C., followed by addition of 9.5 g of ethanolamine. The temperature rises to room temperature over 1.5 hours. After this time, the reaction medium is diluted in 920 ml of water and diafiltration is then performed against 5 volumes of brine (0.9% NaCl) and 8 volumes of water. The polymer solution is then frozen and freeze-dried. 5.2 g of the polymer (2) are obtained, i.e. a yield of 68%. The percentage of C12 graft determined by ¹H NMR in TFA-d is 14.4%. The percentage of hydroxyethylglutamine determined by ¹H NMR in TFA-d is 86.0%. The Mn (determined by GPC NMP) is 22.7 kg/mol as PMMA equivalents.

### Example 2

### Synthesis of Polymer (2), pHEG T

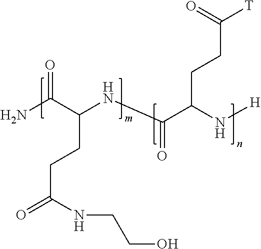

Indices and groups: m=211, n=9, T=D,L-alpha-tocopherol (T)

5 g of a poly(glutamic acid) with a degree of polymerization (DP) of 220 and randomly 4 mol % grafted with synthetic alpha-tocopherol (obtained according to the procedure described in WO-A-03/104 303) are dissolved, by heating to 80° C., in 77 ml of DMF in a 250 ml three-necked round-bottomed flask. 5.3 ml of isobutyl chloroformate and 4.5 ml of N-methylmorpholine are added to this solution cooled to 0° C. The reaction medium is stirred for 15 minutes, followed by addition of 8.3 ml of ethanolamine. The temperature, maintained for 5 minutes at 0° C., then rises to room temperature and the medium is stirred for 2 hours. After this time, the reaction medium is quenched with 2 ml of 1N HCl and then diluted in 600 ml of water, followed by dialysis (1 kD tube) against 1 volume of brine (0.9% NaCl) and 3 volumes of water. The polymer solution is then frozen and freeze-dried. 6.1 g of polymer (3) are obtained, i.e. a yield of 96%. The percentage of tocopherol determined by ¹H NMR in TFA-d is 4.5%. The percentage of hydroxyethylglutamine determined by ¹H NMR in TFA-d is 95%. The Mn (determined by GPC NMP) is 116 kg/mol as PMMA equivalents.

### Example 3

### Synthesis of Polymer C1, PHEG-distearylamine

The comparative polymer C1 was synthesized according to example 4 of patent application WO-A-02/098 952. The polymer contains a distearylamine group at the end of the chain consisting of 40 polyhydroxyethylglutamine units.

### Example 4

### Study of Combination with Insulin

An aqueous solution containing 10 mg of polymer per milliliter at pH 7.4 and 200 IU of insulin (7.4 mg) is prepared. The solutions are incubated for 2 hours at room temperature and the free insulin is separated from the associated insulin by ultrafiltration (threshold at 100 KDa, 15 minutes at 10 000×G at 18° C.). The free insulin recovered in the filtrate is then assayed by HPLC (High-Performance Liquid Chromatography) and the amount of associated insulin is deduced. The results are given in table 1 below.

TABLE 1

| Polymer | % Association |
|---|---|
| 1 | 96 |
| 2 | 98 |
| C1 | 81 |

The results demonstrate that the polymers of the invention are capable of strongly associating insulin to give colloidal suspensions greater than 100 KDa in size and the degrees of association with insulin are very high. The polymer Cl with a hydrophobic distearyl group at the end of the chain is less efficient. The associating capacity of these polymers makes them suitable for use as vectorizing agents.

### Example 5

### Solubility of the Polymer as a Function of the pH

The solubility of polymer 2 was compared with that of the reference polymer, sodium polyglutamate grafted with about 5 mol % of alpha-tocopherol, synthesized as described in patent application WO-A-03/104 303 (polymer C2). The result is as follows:

| Polymer at | Solubility at | Solubility at |
|---|---|---|
| 20 mg/ml | pH 7.4 | pH < 5 |
| Reference polymer C2 | yes | no |
| Polymer 2 | yes | yes |

It appears that the solubility of polymer 2 extends over a wide pH range.

1. A copolyhydroxyalkylglutamine, characterized in that it comprises a plurality of pendent and identical or different hydrophobic groups (HG).

2. The copolyhydroxyalkylglutamine as claimed in claim 1, characterized in that it comprises on average at least 3 hydrophobic groups (HG) per copolymer chain.

3. The copolyhydroxyalkylglutamine as claimed in claim 1 or 2, characterized in that it comprises identical or different hydroxyalkylamine groups preferably chosen from the following groups: 2-hydroxyethylamine, 3-hydroxypropylamine, 2,3-di-hydroxypropylamine, tris(hydroxymethyl)amino-methane and 6-hydroxyhexylamine.

4. The copolyhydroxyalkylglutamine according to any one of the preceding claims, characterized in that the hydrophobic group (HG) contains from 8 to 30 carbon atoms.

5. The copolyhydroxyalkylglutamine as claimed in claim 4, characterized in that the hydrophobic groups HG are chosen from the group comprising:
linear or branched C8 to C30 alkyls possibly containing at least one unsaturation and/or at least one heteroatom,
C8 to C30 alkylaryls or arylalkyls possibly containing at least one unsaturation and/or at least one heteroatom, and
C8 to C30 (poly)cyclics possibly containing at least one unsaturation and/or at least one heteroatom.

6. The copolyhydroxyalkylglutamine as claimed in any one of the preceding claims, characterized in that at least one of the hydrophobic groups HG is obtained by grafting, starting with a precursor chosen from the group comprising: octanol, dodecanol, tetradecanol, hexadecanol, octadecanol, oleyl alcohol, tocopherol or cholesterol.

7. The copolyhydroxyalkylglutamine as claimed in any one of the preceding claims, characterized in that it comprises alpha-L-glutamate units and/or alpha-L-glutamic units.

8. The copolyhydroxyalkylglutamine as claimed in any one of the preceding claims, characterized in that it corresponds to one of the general formulae (I) below: 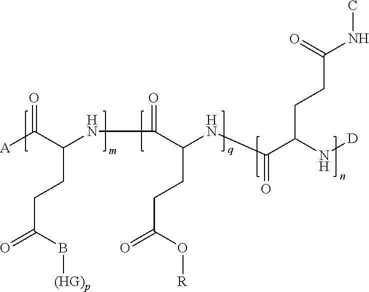
in which
A independently represents:
a group NHR² in which R² represents an H, a linear C2 to 010 or branched C3 to C10 alkyl or a benzyl,
a terminal amino acid unit linked via the nitrogen and the acid function(s) of which is (are) optionally modified with an amine or an alcohol corresponding to the definitions NHR² and OR², respectively;
B is a divalent, trivalent or tetravalent bonding group preferably chosen from the following radicals:
—O—, —NH—, —N—(C1 to C5)alkyl-, an amino acid residue (preferably of a natural amino acid), a diol, a triol, a diamine, a triamine, an amino alcohol or a hydroxy acid containing from 1 to 6 carbon atoms;
C is a mono-, di or trihydroxy(C1 to C6)alkyl group, preferably hydroxyethyl, hydroxypropyl or trishydroxymethylmethane;
D represents an H, a linear C2 to 010 or branched C3 to 010 acyl group or a pyroglutamate;
the hydrophobic groups HG represent, independently of each other, a radical chosen from:
linear or branched C8 to C30 alkyls possibly containing at least one unsaturation and/or at least one heteroatom (preferably O and/or N and/or S), or
C8 to C30 alkylaryls or arylalkyls possibly containing at least one unsaturation and/or at least one heteroatom (preferably O and/or N and/or S), or
C8 to C30 (poly)cyclics possibly containing at least one unsaturation and/or at least one heteroatom (preferably O and/or N and/or S);
R represents an H or a cationic species preferably selected from the group comprising:
metallic cations advantageously chosen from the subgroup comprising: sodium, potassium, calcium, magnesium;
organic cations advantageously chosen from the subgroup comprising:
amine-based cations,
oligoamine-based cations,
polyamine-based cations (polyethyleneimine being particularly preferred),
amino acid-based cations advantageously chosen from the class comprising cations based on lysine or arginine,
or cationic polyamino acids advantageously chosen from the subgroup comprising polylysine or oligolysine;
m, n and q are positive integers;
(m)/(m+q+n) is defined as the molar degree of grafting of the hydrophobic groups HG and ranges from 0.5 up to 90 mol % on condition that each copolymer chain contains on average at least 3 hydrophobic grafts;
(m+q+n) ranges from 10 to 1000 and preferably between 30 and 500;
(q)/(m+q+n) ranges from 0 to 60 mol %;
p is an integer ranging from 1 to 3.

9. The copolyhydroxyalkylglutamine as claimed in any one of the preceding claims, characterized in that the hydrophobic groups HG are randomly distributed.

10. The copolyhydroxyalkylglutamine as claimed in any one of the preceding claims, characterized in that its molar mass is between 2000 and 200 000 g/mol and preferably between 5000 and 100 000 g/mol.

11. The copolyhydroxyalkylglutamine as claimed in any one of the preceding claims, characterized in that it bears at least one graft of polyalkylene (preferably ethylene) glycol type linked to a glutamate unit.

12. A pharmaceutical, cosmetic, dietetic or plant-protection composition comprising at least one copolyhydroxyalkylglutamine as claimed in any one of claims 1 to 11.

13. The composition as claimed in claim 12, characterized in that it comprises at least one active principle.

14. A composition, especially as claimed in claim 13, characterized in that the active principle is combined with the copolyhydroxyalkylglutamine(s) via one or more bonds other than one (or more) covalent chemical bond(s).

15. The composition as claimed in claim 13 or 14, characterized in that the active principle is a protein, a glycoprotein, protein linked to one or more polyalkylene glycol chains, a polysaccharide, a liposaccharide, an oligonucleotide, a polynucleotide or a peptide.

16. The composition as claimed in claim 13 or 14, characterized in that the active principle is a small hydrophobic, hydrophilic or amphiphilic organic molecule.

17. The composition as claimed in any one of claims 12 to 16, characterized in that it may be administered via the oral, parenteral, nasal, vaginal, ocular, subcutaneous, intravenous, intramuscular, intradermal, intraperitoneal, intracerebral or buccal route.

18. The composition as claimed in any one of claims 12 to 17, characterized in that it is in the form of a gel, a solution, an emulsion, micelles, nanoparticles, microparticles, a powder or a film.

19. The composition as claimed in any one of claims 12 to 18, characterized in that it is a colloidal suspension of nanoparticles and/or microparticles and/or micelles of copolyhydroxyalkylglutamines in an aqueous phase.

20. The composition as claimed in any one of claims 12 to 19, characterized in that it is in the form of a solution in a biocompatible solvent and in that it may be injected subcutaneously, intramuscularly or into a tumor.

21. The composition as claimed in claim 20, characterized in that it is capable of forming a deposit at the site of injection.

22. A process for preparing medicaments, in particular for oral, nasal, vaginal, ocular, subcutaneous, intravenous, intramuscular, intradermal, intraperitoneal or intracerebral administration, the active principles of these medicaments possibly being, especially, proteins, glycoproteins, proteins linked to one or more polyalkylene glycol chains, peptides, polysaccharides, liposaccharides, oligonucleotides, polynucleotides and small hydrophobic, hydrophilic or amphiphilic organic molecules;
and/or nutrients;
and/or cosmetic or plant-protection products;
characterized in that it consists essentially in using at least one copolyhydroxyalkylglutamine as claimed in any one of claims 1 to 11 and/or the composition as claimed in any one of claims 12 to 21.
